(19) United States
(12) Patent Application Publication
(10) Pub. No.: US 2005/0272887 A1
(43) Pub. Date: Dec. 8, 2005

(54) Fluorosulphonated nitrile crosslinkable elastomers based on vinylidene fluoride with low Tg and processes for preparing the same

(75) Inventors: Bruno Michael Ameduri, Montpellier (FR); Abdellatif Manseri, Montpellier (FR); Mario Boucher, Quebec (CA)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: HYDRO-QUEBEC, Montreal (CA)

(21) Appl. No.: 11/198,291

(22) Filed: Aug. 8, 2005

Related U.S. Application Data
(63) Continuation of application No. 10/432,957, filed on Nov. 6, 2003.

(30) Foreign Application Priority Data
Dec. 20, 2000 (CA) 2,328,433

(51) Int. Cl. C07C 255/07; C08F 126/00

(52) U.S. Cl. 526/248; 558/461

(57) ABSTRACT

Monomers corresponding to the formula Z₂C═CWX(CY₂)ₙCN, in which X represents an atom of oxygen or no atom, Z and Y represent an atom of hydrogen or fluorine, W represents an atom of hydrogen or fluorine, W represent an atom of hydrogen or fluorine or a CF₃ group and n is a natural integer between 0 and 10 inclusively. These monomers enable by means of novel copolymerization methods to prepare fluorosulphonated nitrite elastomers having very low glass transition temperatures (T_{g}).

### CROSS-REFERENCE TO RELATED APPLICATIONS

This application is a continuation of U.S. application Ser. No. 10/432,957, which was the National Stage of International Application No. PCT/CA01/01439, filed on Oct. 12, 2001, which claims priority to Canadian Application 2,328,433 filed on Dec. 20, 2000. U.S. application Ser. No. 10/432,957 is hereby expressly incorporated herein by reference.

### SCOPE OF THE INVENTION

The present invention concerns fluorosulphonated nitrile crosslinkable elastomers having the specific feature of exhibiting low glass transition temperatures (T_{g}).

The present invention also concerns novel methods making possible, in particular, synthesis of crosslinkable elastomers exhibiting low glass transition temperatures (T_{g}) as well as the use of such elastomers in manufacturing of stable parts intended, in particular, for the aeronautical, petroleum, automotive, mining and nuclear industries as well as in plastics technology. By way of example, such elastomers are useful in the manufacturing of mechanically and chemically stable parts such as membranes, polymer electrolytes, ionomers, fuel cell components supplied e.g. with hydrogen or methanol, sealing gaskets, O-rings, radiator hoses, tubes, pump housings, diaphragms and piston heads.

Because of their chemical inertia, ion exchange membranes that are partially or completely fluorinated are usually chosen in the chlorine-soda type processes or for fuel cells supplied in particular with hydrogen or methanol. Such membranes are available commercially under names such as Nafion®, Flemion®, Dow®. Other similar membranes are proposed by Ballard Inc. in the application WO 97/25369, which describes copolymers, among others, based on tetrafluoroethylene (TFE) and perfluorovinyl ethers.

The present invention also concerns monomer compounds that can be used, in particular, in the synthesis of fluorosulphonated crosslinkable nitrile elastomers.

The term copolymer as it is used in the scope of the present invention relates to compounds formed of macromolecules comprising different monomer units that are 2, 3, 4, 5, 6 or more in number. Such compounds with high molar masses are obtained when one or several monomers polymerize together. By way of example of copolymers thus obtained using 3, 4, 5 or 6 different monomer units are the terpolymers, the tetrapolymers, the pentapolymers and the hexapolymers obtained, respectively, by the reactions of terpolymerization, tetrapolymerization, pentapolymerization and hexapolymerization.

### PRIOR ART

Fluorinated elastomers exhibit a unique combination of properties (resistance to heat, to oxidation, to ultraviolet (UV), to aging, to chemical corrosive agents and to fuels; low surface tension, low index of refraction, low dielectric constant and low water absorption), which has allowed them to be used in “high tech” applications in numerous areas: sealing gaskets (space, aeronautics), semi-conductors (microelectronics), radiator hoses, tubes, pump housings and diaphragms (chemical, automotive and petroleum industries).

Fluorinated elastomers [(Prog. Polym. Sci. 26 (2001) 105-187 and Kaut. Gummi Kunst. 39 (1986) 196] and, in particular, the copolymers and the terpolymers based on vinylidene fluoride (or 1,1-difluoroethylene, VDF, VF2) are the polymers of choice for applications such as coatings and paints and, more recently, membranes or components of fuel cells.

These polymers are resistant to corrosive, reducing or oxidizing conditions as well as to hydrocarbons, solvents and lubricants (Prog. Polym. Sci. 26 (2001) 138-144).

To improve their chemical inertia and mechanical properties, it is necessary to crosslink these elastomers. Elastomers based on VDF can be crosslinked by various means (chemical in the presence of polyamines, polyalcohols and organic or ionizing peroxides or by electron bombardment), described in detail in the reviews Prog. Polym. Sci. 26 (2001) 105, Rubber Chem. Technol. 55 (1982) 1004, and in the work “Modern Fluoropolymers” (1997), chapters 32 (p. 597) and 18 (p. 335). However, it is possible that the products crosslinked using polyamines or polyalcohols do not correspond to the optimum applications that are the goal, e.g. elastomers as sealing gaskets or radiator hoses, diaphragms, pump housings for use in the automotive industry [Casaburo, Caoutchoucs et Plastiques 753 (1996) 69]. Crosslinking using peroxides is more encouraging, above all using fluoroiodated or fluorobromated elastomers.

It is also possible to conceive other methods of crosslinking of fluorinated elastomers: in the presence of salts (e.g. potassium salts) of hydroquinone, of bisphenol A or bisphenol AF to crosslink the elastomers that carry pentafluorophenyl groups [Prog. Polym. Sci. 26 (2001) 157]; using the “thiolene” systems between elastomers that carry mercaptan functions and non-conjugated dienes [Designed Monomers and Polymers 2 (1999) 267] and by crosslinking of elastomers that have nitrile groups using catalytic interactions of tetraalkyltin (and particularly tetraphenyltin) or silver oxide. This latter method is especially interesting since a triazine cycle, that is very stable at high temperature and with respect to oxidizing and to chemical agents, is formed [Prog. Polym. Sci. 26 (2001) 105 and chapter of A. L. Logothetis “Perfluoroelastomers and their Functionalization” in the work by Hafada, Kitayama, Vogl entitled “Macromolecular Design of Polymeric Materials” (1997)].

Thus, the use of new reactive monomers having crosslinkable functions, the “Cure Site Monomers” (CSM), is especially interesting for preparing on request fluorinated elastomers using radical copolymerization.

One of the main interests in the present invention rests in obtaining fluorinated elastomers that carry nitrile groups.

However, the fluorinated nitrile elastomers described in the literature are few in number because of the complexity of monomer synthesis: Breazeale [U.S. Pat. No. 4,281,092 (1981)] and Nakagawa [U.S. Pat. No. 4,499,249 (1985)] describing the syntheses of F₂C═CF[OCF₂CF(CF₃)]ₙO(CF₂)ₘCN and of F₂C═CFOC₄F₈CN in 4 and 6 steps, respectively.

Various companies use trifluorovinyl ethers without nitrile groups but functional, also containing other ether bridges that promote a decrease in the glass transition temperature (T_{g}). These functional monomers have led to industrial products.

For example, the DuPont company markets Nafion® membranes obtained by copolymerization of tetrafluoroethylene (TFE) with the monomer F₂C═CFOCF₂CF(CF₃)OC₂F₄SO₂F (PFSO₂F). In the same way, the Asahi Glass company uses this sulphonated monomer for manufacturing the Flemion® membrane. Other monomers with the same functionality, for example, F₂C═CFOCF₂CF(CF₃)OC₃F₆SO₂F (for the membrane Aciplex®, Asahi Chemical) or F₂C═CFOC₂F₄SO₂F or even with carboxylate functionality such as the monomer F₂C═CFO[CF₂CF(CF₃)O]ₓC₂F₄CO₂CH₃ (for Nafion® or Aciplex® membranes when x is equal to 1, and for Flemion if x is equal to 0) are also used.

This situation prompted us to use VDF (an alkene that is less expensive and easier to process than TFE) in a larger quantity and to conceive its novel copolymerization with nitrile monomers and functional perfluoroalkyl vinyl ethers (PAVE) and/or functional perfluoroalkoxy alkyl vinyl ethers and most particularly PFSO₂F. In fact, on one hand, the work on copolymerization of fluorinated alkenes with perfluorinated vinyl ethers and nitrile monomers utilize only TFE and perfluoromethyl vinyl ether [U.S. Pat. No. 4,281,092 (1981); U.S. Pat. No. 4,972,038 (1990); U.S. Pat. No. 5,677,389 (1997); WO 97/19982 and European patent application 11,853 (1980)] and on the other hand, this monomer is interesting in that it is functional and stimulates the crosslinking sites to produce novel elastomers having good resistance at low temperatures and good aid-processing properties. In addition, the Hydro-Quebec company has described easy copolymerization of PFSO₂F with VDF (in the PCT application referenced WO 01/49757) and terpolymerization of PFSO₂F with VDF and HFP (in the PCT application referenced WO 01/49760). In addition, the use of nitrile monomers promotes crosslinking (by tetraphenyltin or silver oxide) of the polymers formed and improves their thermostability, their mechanical properties and their resistance to chemical agents, to petroleum, to strong acids and to oxidation.

The most related studies concern copolymerization (and terpolymerization with the use of a fluorinated olefin and, in our case, vinylidene fluoride in particular) of trifluorovinyl ethers with nitrile monomers.

It can be noted that the majority of the syntheses based on nitrile monomers and trifluorovinyl ethers involve tetrafluoroethylene (TFE) such as the terpolymers TFE/perfluoro(8-cyano-5-methyl-3,6-dioxa-1-octene) [U.S. Pat. No. 4,281,092] or TFE/perfluoro(4-cyanobutyl vinyl ether) [U.S. Pat. No. 4,499,249]. In addition, in terpolymerization involving a perfluorovinyl ether, perfluoromethyl vinyl ether (PMVE) is essentially used [U.S. Pat. No. 4,281,092 (1981); U.S. Pat. No. 4,972,038 (1990); U.S. Pat. No. 5,677,389 (1997); WO 97/19982 and European patent application 11,853 (1980)].

Syntheses of nitrile monomers are complex and delicate; they require numerous steps. Thus, it was essential to find novel, simple and fast means of synthesis that are able to lead to monomers and thus elastomers that are industrially accessible.

The known literature makes no mention of copolymerization of nitrile olefins with PFSO₂F, nor of the terpolymerization of these two monomers with VDF, which makes up the distinctive character of the present invention.

### SUMMARY OF THE INVENTION

The present invention describes the preparation and the copolymerization, of trifluorovinyl monomers with a terminal nitrile with fluorinated monomers. This process leads to the synthesis of novel fluorosulphonated crosslinkable nitrile elastomers having very low glass transition temperatures (T_{g}), good resistance to acids, to petroleum and to fuels and good aid-processing properties. These elastomers contain, by way of example, from 2 to 14 mol-% of 5,6,6-trifluoro-5-hexene nitrile (F—CN), from 20 to 30 mol-% perfluoro(4-methyl-3,6-dioxaoct-7-ene)sulphonyl fluoride (PFSO₂F) and 66 to 78 mol-% vinylidene fluoride (VDF or VF₂). In this specific case, they are prepared using radical copolymerization of F—CN and PFSO₂F or by radical terpolymerization of F—CN, of PFSO₂F and of VDF in the presence of different organic initiators such as, e.g. peroxides, peresters or diazo compounds. Other fluorinated olefins, such as vinyl fluoride, trifluoroethylene, chlorotrifluoroethylene, hexafluoropropene, 1,2-difluorodichloroethylene, 1,1-difluoro-2-chloroethylene, or 1-hydropentafluoropropene may also be used in the tetrapolymerization. In addition, the present invention concerns a crosslinking process for these polymers.

Finally, the present invention concerns the use of the elastomers thus obtained in varied areas of application, in particular manufacturing of membranes, sealing gaskets and in the plastics industry.

### DETAILED DESCRIPTION OF THE INVENTION

A first object of the present invention comprises by the family of compounds corresponding to formula I:
Z₂C═CWX(CY₂)ₙCN (I)
in which:
   X represents an atom of oxygen or no atom;
   Y represents an atom of hydrogen or fluorine;
   Z represents an atom of hydrogen or fluorine;
   W represents an atom of hydrogen or fluorine or a CF₃ group; and
   n is a natural integer between 0 and 10 inclusively.

According to a preferred embodiment, the present invention comprises the sub-family of compounds corresponding to formula II:
F₂C═CF(CH₂)ₙCN (II)
in which:
   n is a natural integer between 0 and 10 inclusively.

A second object of the present invention consists of a process of preparation for a fluorinated copolymer by radical copolymerization, the said process comprising the reaction of a compound corresponding to formula I:
Z₂C═CWX(CY₂)ₙCN (I)
with a compound corresponding to formula III₁:
   F₂C═CFOR_{F1} (III₁)
in which R_{F1} designates:
   a linear or branched group of the formula CₙF₂ₙ₊₁ (n designates a natural integer varying from 1 to 10); or
with a compound corresponding to the formula III₂:
   F₂C═CFOR_{F2}-G (III₂)
in which R_{F2} designates:
   a linear or branched group of the formula (CF₂CFX′)_{y}[O(CF₂)₁]ₘ wherein X′ represents a fluorine atom or a CF₃ group;
   y, 1 and m are natural integers between 1 and 5, 1 and 4, and 0 and 6 inclusively, respectively; and
in which G represents:
   a functional group SO₂F, CO₂H, CO₂R (wherein R designates the group CₚH₂ₚ₊₁, in which p represents a natural integer varying from 0 to 5) or designates a functional group P(O)(OR′) in which R′ designates, independently, a hydrogen atom or an alkyl group in C₁-C₅.

According to an advantageous embodiment of this process, the fluorinated copolymer is prepared by reaction of a compound corresponding to formula II′:
F₂C═CF(CH₂)₃CN (II′)
with a compound of formula III₁ or a compound of formula III₂, formulas III₁ and III₂ being such as defined above:
   in such a way as to obtain a random copolymer that corresponds to the formula IV: 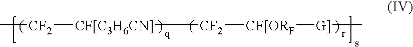
in which:
   R_{F} represents the groups R_{F1} or R_{F2}, the group G being absent when R_{F} represents R_{F1} and the group G, when it is present with R_{F2}, being as defined above; and
in which:
   q, r and s represent, independently, natural integers such that the ratio q/r varies from 1 to 30 and such that s varies from 20 to 300, preferably the ratio q/r varies from 1 to 25 and s varies from 25 to 250, and still more preferably the ratio q/r varies from 3 to 20 and s varies from 30 to 220.

A third object of the present invention consists of a process of copolymerization, comprising the reaction of the compound corresponding to formula II′:

ti F₂C═CF(CH₂)₃CN (II′)

with a compound corresponding to formula II₁:
   F₂C═CFOR_{F1} (III₁)
in which R_{F1} designates:
   a linear or branched group of the formula CₙF₂ₙ₊₁ (n designating a natural integer varying from 1 to 10); or
with a compound corresponding to the formula III₂:
   F₂C═CFOR_{F2}-G (III₂)
in which R_{F2} designates:
   a linear or branched group of the formula (CF₂CFX′)_{y}[O(CF₂)₁]ₘ wherein X′ represents a fluorine atom or a CF₃ group;
   y, 1 and m are natural integers between 1 and 5, 1 and 4, and 0 and 6 inclusively, respectively; and
in which G represents:
   a functional group SO₂F, CO₂H, CO₂R (with R designating the group CₚH₂ₚ₊₁, in which p represents a natural integer varying from 0 to 5) or designating a functional group P(O)(OR′) in which R′ designates, independently, a hydrogen atom or an alkyl group in C₁-C₅ and
      with a compound of formula V:
      FCX═CYZ (V)
in which:
   X, Y and Z represent, independently, atoms of hydrogen, fluorine, chlorine or groups with the formula CₙF₂ₙ₊₁ (n equaling 1, 2 or 3) but in no case X=Y=Z=F,
      in such a way as to obtain a random copolymer corresponding to formula VI: 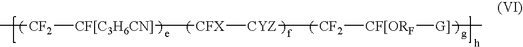
in which:
   R_{F} represents the groups R_{F1} or R_{F2} previously defined, group G being absent when R_{F} represents R_{F1}; and
in which:
   e, f, g and h represent, independently, natural integers such that the ratio f/e varies from 5 to 50, such that the ratio f/g varies from 1 to 20 and such that h varies from 10 to 250, preferably the ratio f/e varies from 5 to 30, the ratio f/g varies from 2 to 10 and h varies from 15 to 200, and still more preferably the ratio f/e varies from 10 to 25, the ratio f/g varies from 2 to 5 and h varies from 20 to 150.

According to an advantageous embodiment of the processes defined above, the copolymerization reaction is carried out in batch and preferably this reaction is carried out in emulsion, in microemulsion, in suspension or in solution.

The reaction is preferably initiated in the presence of at least one organic radical initiator preferably chosen from the group made up of alkyl peroxides, peresters, percarbonates, alkyl peroxypivalates and diazo compounds.

In a particularly advantageous manner, the reaction is carried out in the presence:
of at least one peroxide, preferably chosen from the group consisting of t-butyl peroxide, t-butyl hydroperoxide, t-butyl peroxypivalate and t-amyl peroxypivalate; and/or
of at least one perester which is preferably benzoyl peroxide; and/or
at least one percarbonate that is preferably t-butyl cyclohexyl peroxydicarbonate.

Preferably, the concentration of peroxide and/or perester and/or percarbonate in the reaction medium is such that the initial molar ratio between initiator and the monomers ([initiator]₀/[monomers]₀) is between 0.1 and 2%, preferably between 0.5 and 1%, the initiator being the compound of the formula tBuO-OtBu or tBuO-OC(O)tBu and the monomers being compounds of formula I, II, III₁, III₂, II′ and V; the expression [initiator]₀ expresses the initial molar concentration of initiator and the expression [monomers]₀ expresses the total initial concentration of monomers.

According to another advantageous embodiment of the processes of the invention, the reaction is carried out:
in the presence of t-butyl peroxypivalate and at a reaction temperature between 70 and 80° C., preferably at a temperature of around 75° C.; or
in the presence of t-butyl peroxide and at a reaction temperature between 135 and 145° C., preferably at a temperature of around 140° C.

Advantageously, the reaction is carried out in solution in the presence of at least one organic solvent. This organic solvent is preferably chosen from the group consisting of perfluoro-n-hexane, acetonitrile or mixtures of perfluoro-n-hexane and acetonitrile and the amount of solvent in the reaction mixture is such that the initial mass ratio between the solvent and the monomers is between 0.5 and 1.5, and preferably between 0.6 and 1.2.

According to a particular embodiment of the invention, the reagent of formula III₂ is perfluoro(4-methyl-3,6-dioxaoct-7-ene)sulphonyl fluoride and the compound of formula V is vinylidene fluoride.

A fourth object of the present invention consists of the family of fluorinated polymers, preferably fluorinated copolymers, and more preferably the family of fluorosulphonated nitrile copolymers that can be obtained by using the processes that are the object of the present invention.

A preferable sub-family of fluorosulphonated nitrile copolymers that are the object of the present invention consists of copolymers containing:
from 1 to 20% of 5,6,6-trifluoro-5-hexene nitrile (F—CN);
from 20 to 33% perfluoro(4-methyl-3,6-dioxaoct-7-ene)sulphonyl fluoride (PFSO₂F); and
from 65 to 79% vinylidene fluoride (VDF),
   the percentages being expressed in moles.

Still more preferably among the fluorosulphonated nitrile copolymers are those containing:
2 to 14% of 5,6,6-trifluoro-5-hexene nitrile (F—CN);
from 20 to 30% of perfluoro(4-methyl-3,6-dioxaoct-7-ene)sulphonyl fluoride (PFSO₂F); and
from 66 to 78% of vinylidene fluoride (VDF),
   the percentages being expressed in moles.

Typical fluorosulphonated nitrile copolymers according to the present invention exhibit spectroscopic characteristics identical or similar to those illustrated in Table 2 below.

A fifth object of the present invention consists of a process permitting the preparation of a fluorosulphonated nitrile elastomer according to the present invention. This process consists of submitting one or several copolymers according to the invention to a crosslinking step, preferably carried out in the presence of tetraphenyltin or silver oxide in proportions varying from 0.1 to 10 parts by weight for 100 parts by weight of fluorosulphonated nitrile copolymer, the mixture being pressed (pressure of 20 bars) at 175° C. for 2 hours, then at 200° C. for 24 hours, and finally at 220° C. for 12 hours.

The sixth object consists of fluorosulphonated nitrile elastomers that can be obtained by a process according to the fifth object of the present invention.

Among the fluorosulphonated nitrile elastomers according to the invention, those having very low glass transition temperatures (T_{g}) are of particular interest. More particularly those that have a glass transition temperature, measured according to the standard ASTM E-1356-98, preferably between −43 and −22° C., still more preferably between −34 and −29° C., offer interesting application possibilities in the areas of high technology.

Among these elastomers, a preferable sub-family consists of those that exhibit an inherent viscosity, measured according to the method ASTM D-2857-95, between 0.9 and 2.0 ml/g and/or that exhibit a thermostability ATG up to 297° C. in air at 10C/min., the temperature value at which a loss of mass of 5% is measured.

A seventh object of the present invention consists of the use of one or several of the fluorosulphonated crosslinkable nitrile elastomers according to the invention for:
manufacturing membranes, polymer electrolytes, ionomers, fuel cell components supplied e.g. with hydrogen or methanol;
obtaining sealing gaskets and O-rings, radiator hoses, tubes, pump housings, diaphragms, piston heads (for applications in the aeronautical, petroleum, automotive, mining, nuclear industries); and
for the plastics industry (products that aid processing).

An eighth object of the present invention consists of a process for crosslinking the sulphonyl groups of a sulphonated polymer chosen from the family of fluorosulphonated nitrile elastomers according to the sixth object, in which:
said polymer is brought into contact with a crosslinking agent that makes reaction possible between two sulphonyl groups coming from adjacent polymer chains to form said crosslinking bonds; and
at least one fraction of the bonds formed at the time of crosslinking has an ionic charge.

Thus, the invention describes, in particular, the synthesis of novel fluorinated copolymer elastomers with a base of synthetic fluorinated nitrile comonomers (such as F—CN) and containing a functional perfluoroalkyl vinyl ether and/or a functional perfluoroalkoxyalkyl vinyl ether and possibly other fluorinated alkenes. The originality of this invention rests basically on the following facts:
1) The preparation of ω-nitrile trifluorovinyl monomers, reactive in copolymerization with commercial fluorinated alkenes or functional fluorinated monomers;
2) The synthesis of fluorinated elastomers based on functional perfluoroalkyl vinyl ethers and/or functional perfluoroalkoxy vinyl ethers and possibly other fluorinated alkenes is carried out with VDF instead of tetrafluoroethylene (TFE), the latter being largely used for manufacturing fluorinated elastomers;
3) The synthesis of fluorinated elastomers which are involved in this invention does not require the use of monomers that carry siloxane groups, the latter generally contributing to a decrease in the glass transition temperature (T_{g}) but having the disadvantage of beginning to degrade from 200° C.

4) The crosslinkable fluorinated elastomers obtained by the present invention have, as the minority of their composition, fluorinated nitrile monomers with the structure Z₂C═CWX(CY₂)ₙCN (wherein X, Y, Z, W and n defined as above) and, as the majority, functional perfluoroalkyl vinyl ether (PAVE) or functional perfluoroalkoxyalkyl vinyl ether (PAAVE) for the copolymers; and for the terpolymers, as the minority of the composition, fluorinated nitrile monomers and, as the majority, VDF or functional perfluoroalkyl vinyl ether or functional perfluoroalkoxyalkyl vinyl ether, depending on the initial molar ratios of these two fluorinated monomers.

5) The fluorinated elastomers synthesized by said invention have very low glass transition temperatures (T_{g}), these elastomers can thus be used in applications in the area of the plastics industry (“Aid Processing”) or other high technology industries (aerospace, electronics, automotive, petroleum, transport of fluids that are corrosive, acid or very cold such as liquid nitrogen, oxygen and hydrogen). In addition, sealing gaskets with high thermal resistance can be produced using these elastomers;

6.) The fluorosulphonated nitrile elastomers are easily crosslinked in the presence of tetraalkyltin or tetraphenyltin. This crosslinking significantly improves the properties of resistance to heat, to oxidation, to solvents, to hydrocarbons, to fuels, to acids and to aggressive media.

In addition, it is well known that the perfluorinated polymers cannot usually be crosslinked using the techniques that are traditionally used for nonfluorinated polymers because of easy elimination of the fluoride ion and the steric hindrance of the perfluorinated chains. However, the technique generally described in the application PCT WO 99/38897, of which the contents are incorporated by reference, makes it possible to create crosslinks, i.e. bonds, between the sulphonyl groups attached to the adjacent polymer chains, including those having a perfluorinated skeleton, e.g. those derivatives of the following monomer and its copolymers: 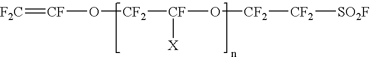
in which X is F, Cl or CF₃; and
n is 0 to 10 inclusively.

Advantageously, the crosslinking can thus be carried out while the polymer is in the form of the nonionic polymer precursor, but after having been moulded or pressed into the desired shape. A material thus results that is much more mechanically resistant. The present invention also concerns the moulding or pressing of the crosslinked polymer in the form of membranes or hollow fibres (hereinafter referred to as “membranes”) for use in a fuel cell, an electrolyzer in water, a chlorine-soda process, electrosynthesis, water treatment and production of ozone. The use of crosslinked polymers as initiators of certain chemical reactions due to the great dissociation of the ionic groups introduced by the crosslinking technique and the insolubility of the polymer chain is also part of the invention.

Creation of stable crosslinks is carried out by mediating a reaction between two —SO₂L groups coming from adjacent polymer chains. The reaction is initiated by a crosslinking agent and permits the formation of derivatives according to the following formulas: 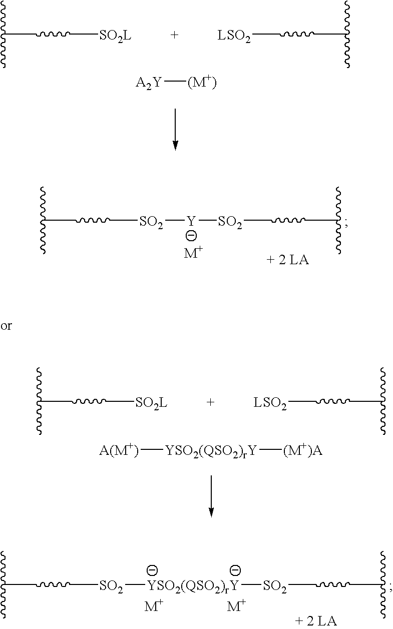
in which:
   r is or 1;
   M comprises an inorganic or organic cation;
   Y comprises N or CR in which R comprises substituted or non-substituted H, CN, F, SO₂R³, C₁₋₂₀ alkyl; substituted or non-substituted C₁₋₂₀ aryl; substituted or non-substituted C₁₋₂₀ alkylene, in which the substituent comprises one or several halogen atoms and in which the chain comprises one or several F, SO₂R, aza, oxa, thia or dioxathia substituents;
R³ comprises F, substituted or non-substituted C₁₋₂₀ alkyl; substituted or non-substituted C₁₋₂₀ aryl; substituted or non-substituted C₁₋₂₀ alkylene, in which the substituent comprises one or several halogen atoms;
Q comprises a divalent radical C₁₋₂₀ alkyl, C₁₋₂₀ oxaalkyl, C₁₋₂₀ azaalkyl, C₁₋₂₀ thiaalkyl, C₁₋₂₀ aryl or C₁₋₂₀ alkylaryl, each possibly being optionally substituted with one or several halogen atoms and in which the chain comprises one or more oxa, aza or thia substituents;
A comprises M, Si(R′)₃, Ge(R′)₃ or Sn(R′)₃ in which R′ is C₁₋₁₈ alkyl;
L comprises a labile group such as a halogen atom (F, Cl, Br), an electrophilic heterocycle N-imidazolyl, N-triazolyl, R²SO₃ in which R² is an optionally halogenated organic radical; and
R² comprises the proton; the alkyl, alkenyl, oxaalkyl, oxaalkenyl, azaalkyl, azaalkenyl, thiaalkyl, thiaalkenyl, dialkylazo, optionally hydrolyzed silaaklyl radicals, optionally hydrolyzed silaalkenyls, said radicals being linear, branched or cyclic and comprising from 1 to 18 carbon atoms; the aliphatic cyclic or heterocyclic radicals with 4 to 26 carbon atoms optionally comprises at least one lateral chain comprising one or several heteroatoms such as nitrogen, oxygen or sulphur; the aryls, arylalkyls, alkylaryls and alkenylaryls with 5 to 26 carbon atoms optionally including one or several heteroatoms in the aromatic nucleus or in a substituent.

The crosslinking reaction may involve all of the sulphonyl groups or only a fraction of them. The crosslinking reagents may be added or used according to different techniques well known to the person skilled in the art. Advantageously, the polymer is moulded into the desired form before crosslinking, e.g. in the form of membranes or hollow fibres, and the material is immersed in or covered with a solution of the crosslinking agent in one or several solvents that promote the coupling reaction.

If only a fraction of the bonds forming the bridge between the polymer chains is required, the remaining —SO₂L groups can be hydrolyzed in a conventional manner in the form of sulphonate by alkaline hydrolysis.

The crosslinked polymer obtained according to the process of the present invention can be easily separated from the secondary reaction products which are, e.g. volatile, such as (CH₃)₃SiF or (CH₃)₃SiCl. Alternatively, the crosslinked polymer can be washed using an appropriate solvent like water or an organic solvent in which it is insoluble. In addition, classic techniques well known to the person skilled in the art, for example ion exchange or electrophoresis, can be used to change the cation M⁺ obtained in the crosslinking reaction and/or coming from the non-crosslinking ionogenic agent by using the desired cation for the final application.

The advantages related with the present invention are basically the following:
1) Use of commercial nitrile olefins and/or preparation of novel fluorinated nitrile monomers by simple synthetic means;
2) Fluorinated nitrile monomers that are reactive in copolymerization are used;
3) The synthesis process is implemented in batch mode;
4) The process that this invention involves is carried out in solution and uses classic organic solvents that are easily available commercially;
5) The process of said invention consists of a radical polymerization in the presence of classic initiators that are easily available commercially;
6) Tetrafluoroethylene (TFE) is not used in this invention. It involves a significant reduction in the costs of synthesis of the copolymers;
7) The perfluorinated olefin that goes into the composition of the fluorinated elastomers prepared by said invention is vinylidene fluoride (VDF); this is clearly less costly and much less dangerous than TFE and gives the elastomers obtained good resistance to oxidation, to chemical agents, to polar solvents and to petroleum and a decrease in the glass transition temperature (T_{g});
8) The fluorinated elastomers that are involved in the invention may be prepared using the PFSO₂F monomer of which copolymerization with acrylonitrile or 5,6,6-trifluoro-5-hexene nitrile (F—CN) and terpolymerization with acrylonitrile or F—CN and VDF have never been the object of works described in the literature. In addition, this monomer sulphonated by means of its sulphonyl fluoride function (—SO₂F) makes it possible to create crosslinking sites in these elastomers;
9) The fluorinated elastomers obtained by this method have very low glass transition temperatures (T_{g}), varying from −43 to −22° C.
10) These fluorosulphonated nitrile copolymers can easily be crosslinked by means of tetraphenyltin or silver oxide, thus leading to materials that are stable, inert and insoluble in all solvents, hydrocarbons or strong acids.

The present invention more particularly concerns the synthesis of reactive ω-nitrile trifluorovinyl monomers and obtaining fluorosulphonated nitrile elastomers based on VDF and PAVE, then the study of their crosslinking as well as their range of application. The crosslinking of these fluorosulphonated nitrile polymers is carried out in the presence of tetraphenyltin or silver oxide, leading to stable triazine cycles. To the best of our knowledge, no study has described the copolymerization of PFSO₂F with monomers having nitrile terminal or terpolymerization of PFSO₂F with nitrile monomers and other fluorinated olefins.

### Synthesis of ω-nitrile trifluorovinyl monomers

The first aspect of this invention consists of making available new trifluorovinyl monomers that are reactive in copolymerization with fluorinated olefins and have a nitrile terminal. The compounds in question can be represented, by way of example, by formulas I and II below:
H₂C CHX(CH₂)ₙCN (I)
F₂C═CFX(CY)ₙCN (II)
in which:
X represents an oxygen atom or no atom;
Y represents an atom of hydrogen or fluorine;
n is a natural integer between 0 and 10, inclusively.

More particularly, the present invention describes compounds corresponding to formulas III and IV:
H₂C═CH(CH₂)ₙCN (III)
F₂C═CF(CH₂)ₙCN (IV)
in which n is such as defined above.

For example, the synthesis of 5,6,6-trifluoro-5-hexene nitrile (F₂C═CFC₃H₆CN) is carried out according to the following reaction diagram: 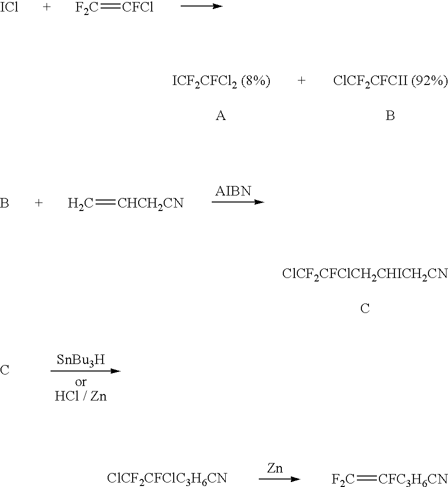
Preparation of Fluorosulphonated Nitrile Elastomers

The field of the present invention extends to all types of processes generally used and especially polymerization in emulsion, in microemulsion, in bulk, in suspension and in solution. However, polymerization in solution is preferably used.

The various fluorinated alkenes used have at the most four carbon atoms and have the structure R₁R₂C═CR₃R₄ wherein the substituents R₁ are such that at least one of the R₁ is fluorinated or perfluorinated. This thus includes: vinyl fluoride (VF), vinylidene fluoride (VDF), trifluoroethylene, chlorotrifluoroethylene (CTFE), 1-hydropentafluoro-propylene, hexafluoroisobutylene, 3,3,3-trifluoropropene, 1,2-difluoro-1,2-dichloroethylene, 1,1-difluoro-2-chloroethylene and, in a general manner, all of the fluorinated or perfluorinated vinyl compounds. In addition, perfluorovinyl ethers also play the role of comonomers. Among them, it is possible to mention the perfluoroalkyl vinyl ethers (PAVE) of which the alkyl group has from one to three carbon atoms: for example, perfluoromethyl vinyl ether (PMVE), perfluoroethyl vinyl ether (PEVE) and perfluoropropyl vinyl ether (PPVE). These monomers can also be perfluoroalkoxy alkyl vinyl ethers (PAAVE), described in the U.S. Pat. No. 3,291,843 and in the reviews Prog. Polym. Sci., A. L. Logothetis, vol. 14 (1989) 251, B. Ameduri et al., vol. 26 (2001) 105, such as perfluoro(2-n-propoxy)propyl-vinyl ether, perfluoro(2-methoxy)propyl-vinyl ether, perfluoro(3-methoxy)propyl-vinyl ether, perfluoro(2-methoxy)ethyl-vinyl ether, perfluoro(3,6,9-trioxa-5,8-dimethyl)dodeca-1-ene, perfluoro(5-methyl-3,6-dioxo)-1-nonene. In addition, the perfluoroalkoxyalkyl vinyl ether monomers with terminal carboxylics or with terminal sulphonyl fluorides such as perfluoro(4-methyl-3,6-dioxaoct-7-ene)sulphonyl fluoride can also be used for the synthesis of fluorinated elastomers described in this invention. Mixtures of PAVE and PAAVE may be present in the copolymers.

More specifically, perfluoro(4-methyl-3,6-dioxaoct-7-ene)sulphonyl-fluoride (PFSO₂F) is used as the comonomer.

The nitrile monomers used in this invention are olefins, in which at least one of the hydrogen atoms has been replaced by a nitrile group and in an optional manner, one or several of the remaining hydrogen atoms have been replaced by an atom of another halogen, essentially fluorine. Certain of these monomers are marketed, such as acrylonitrile, allyl cyanide, alphafluoroacrylonitrile, 1,1-dicyanoethylene or synthesized, such as perfluoro(4-cyanobutyl-vinyl ether) or perfluoro(8-cyano-5-methyl-3,6-dioxa-1-octene), 1,1,2-trifluoro-4-cyanobutene or any other perfluroated carbonitrile monomer. We have also synthesized 5,6,6-trifluoro-5-hexene nitrile.

The solvents used to carry out polymerization in solution are the following:
esters of the formula R—COO—R′ wherein R and R′ are hydrogenated or alkyl substituents that can contain 1 to 5 carbon atoms, but also hydroxy OH groups or ester groups OR″, wherein R″ is an alkyl containing from 1 to 5 carbon atoms, and most particularly wherein R═H or CH₃ and R′═CH₃, C₂H₅, iC₃H₇ and t-C₄H₉;
the fluorinated solvents of the type: ClCF₂CFCl₂, C₆F₁₄, n-C₄F₁₀, perfluoro-2-butyltetrahydrofurane (FC 75); and
acetone, 1,2-dichloroethane, isopropanol, tertiobutanol, acetonitrile or butyronitrile;

The solvents preferably employed are methyl acetate and acetonitrile in variable quantities.

The reaction temperature range can be determined by the decomposition temperature of the initiator; it varies from 20 to 200° C. According to an advantageous embodiment of the processes of the invention, the reaction is carried out:
in the presence of t-butyl peroxypivalate and at a reaction temperature between 70 and 80° C., preferably at a temperature of around 75° C.; or
in the presence of t-butyl peroxide and at a reaction temperature between 135 and 145° C., preferably at a temperature of around 140° C.

In the process according to the invention, it is possible to initiate the polymerization with the use of initiators that are usually used for radical polymerization.

Representative examples of such initiators are azo derivatives (such as azobisisobutyronitrile, AIBN), dialkyl peroxydicarbonates, acetylcyclohexane sulphonyl peroxide, aryl or alkyl peroxide such as dibenzoyl peroxide, dicumyl peroxide, t-butyl peroxide, t-alkyl perbenzoates and t-alkyl peroxypivalates. Still, preference is given to the dialkyl peroxides (preferably t-butyl peroxide), to the dialkyl peroxydicarbonates, such as the diethyl peroxydicarbonates and di-isopropyl peroxydicarbonates and to the t-alkyl peroxypivalates such as t-butyl and t-amyl peroxypivalates and, most particularly, to the t-alkyl peroxypivalates.

For the process of polymerization in emulsion, we used a large range of cosolvents, used in various proportions in mixture with water. In the same way, various surface active agents were used. One of the polymerization processes used can also be by microemulsion as described in European patent EP 250.767 or by dispersion as indicated in U.S. Pat. No. 4,789,717 or the European patents 196.904; 280.312 and 360.292.

The reaction pressures vary between 2 and 120 bars, depending on the experimental conditions.

Chain transfer agents can generally be used to regulate and basically decrease the molar masses of the copolymers. Among these, it is possible to mention telogens containing from 1 to 10 carbon atoms and having terminal atoms of bromium or iodide, such as e.g. compounds of the type R_{F}X (wherein R_{F} is a perfluorated group of the formula CₙF₂ₙ₊₁, n=1 to 10, X designating an atom of bromine or iodine) or X R_{F}′X (with R_{F}′=(CF₂)ₙ wherein n=1 to 6) or alcohols, ethers or esters. A list of the various transfer agents used in telomerization of fluorinated monomers is indicated in the review “Telomerization Reactions of Fluoroalkanes,” B. Améduri and B. Boutevin in the work “Topics in Current Chemistry” (edited by R. D. Chambers), Vol. 192 (1997), p. 165, Springer Verlag 1997.

The entire range of relative percentages of various copolymers that can be synthesized from the fluorinated monomers used and leading to the formation of the fluorinated copolymers, has been studied (Table 1).

The products have been analyzed using 1H and ¹⁹F NMR in acetone or deuterized DMF. This analysis method has made it possible to know, without ambiguity, the percentages of the comonomers introduced into the products. For example, we have completely established, using the microstructures characterized in the literature, the relationships between the characteristic signals of the copolymers VDF/PFSO₂F/F—CN (Table 2) in ¹⁹F NMR and the structure of the products ([Polymer 28 (1987) 224, J. Fluorine Chem. 78 (1996) 145], [PCT application with reference number WO 01/49757], [PCT application with reference number WO 01/49760] and [CA 2,312,194]). This analysis has given evidence of the diads F—CN/PFSO₂F, VDF/PFSO₂F and F—CN/VDF, as well as the head-tail and head-head chaining of the VDF unit blocks (at −91 and −113, −116 ppm, respectively).

The molar percentages of the different monomers contained in the VDF/PFSO₂F/F—CN copolymers have been determined using equations 1, 2 and 3 indicated below (Table 2).

$$\begin{matrix}molar \% of VDF = \frac{A / 2}{A / 2 + B + C} & Equation 1 \\ molar \% of {PFSO}_{2} F = \frac{B}{A / 2 + B + C} & Equation 2 \\ molar \% of F \text{-} CN = \frac{C}{A / 2 + B + C} & Equation 3\end{matrix}$$

in which:
A=L₈₃+L₉₁+L₉₂+L₉₃+L₉₅+L₁₀₈+L₁₁₀+L₁₁₃+L₁₆+L₁₂₇
B=L₁₄₄
C=L_{161 to -165}+L_{178 to -182}
wherein Lᵢ is the value of the signal integration located at −i ppm on the ¹⁹F NMR spectrum.

Using differential calorimetric analysis (DSC), we note that the copolymers are amorphous, exhibit a unique glass transition temperature (T_{g}) and an absence of melting temperature (Table 1). These low values of T_{g} indicate an increased elastomeric character, which is particularly novel for fluorinated nitrile polymers.

In parallel, the thermal stability (ATG), evaluated in air, of these fluorosulphonated nitrile copolymers is very satisfactory.

TABLE 1
Operating conditions and results of radical copolymerizations of VDF with PFSO₂F and F—CN

| | | | | Solvent | | | | | | | | | | | | |
|---|---|---|---|---|---|---|---|---|---|---|---|---|---|---|---|---|
| | Mass | Mass | Mass | (aceto- | | VDF | PFSO₂F | F—CN | VDF | PFSO₂F | F—CN | Conversion rate | | Mass | | T_{deg} |
| | VDF | PFSO₂F | F—CN | nitrile) | C₀ | initial | initial | initial | copo. | copo. | copo. | PFSO₂F | gas | Yield | T_{g} | 5% in air |
| Test | (g) | (g) | (g) | (g) | (%) | (mol-%) | (mol-%) | (mol-%) | (mol-%) | (mol-%) | (mol-%) | (%) | (%) | (%) | (° C.) | (° C.) |
| 5 | 14.0 | 28.4 | 4.6 | 30.0 | 0.5 | 70.3 | 20.0 | 9.7 | 72.0 | 25.0 | 3.0 | 80 | 80 | 75 | −31 | 285 |
| | | | | | t-Bu | | | | | | | | | | | |
| 6 | 20.5 | 29.5 | 5.9 | 32.3 | 0.5 | 72.5 | 15.5 | 9.3 | 78.3 | 18.9 | 2.8 | 71 | 38 | 37 | −29 | 297 |
| | | | | | P.P. | | | | | | | | | | | |
| 7 | 20.3 | 30.8 | 7.1 | 30.5 | 0.5 | 73.1 | 15.9 | 11.0 | 75.8 | 18.3 | 5.9 | 83 | 75 | 74 | −34 | 277 |
| | | | | | t-Bu | | | | | | | | | | | |

P.P = t-butyl peroxypivalate
t-Bu = t-butyl peroxide
Temperature of 74° C. with t-butyl peroxypivalate initiator and 135° C. with t-butyl peroxide initiator
Duration of 15 hours
C₀ = [initiator]₀/([VDF]₀ + [PFSO₂F]₀ + [F—CN]₀)

TABLE 2
¹⁹F NMR Characterization of copolymers VDF/PFSO₂F/F—CN

| | Chemical shift |
|---|---|
| Structure | (ppm) |
| —SO₂F | +45 |
| —OCF₂CF(CF₃)OCF₂CF₂SO₂F | −77 to −80 |
| tBuO—CF₂CH₂— | −83 |
| —CH₂CF₂—CH₂CF₂—CH₂CF₂— | −91 |
| —CF₂CF(R_{F})—CH₂CF₂—CH₂CF₂— | −92 |
| —CF₂CF(R_{F})—CH₂CF₂—CH₂CF₂—CF₂CF(R_{F})— | −93 |
| —CH₂CF₂—CH₂CF₂—CF₂CH₂— | −95 |
| —CF₂CF(OR_{F}SO₂F)—CH₂CF₂—CF₂CF(OR_{F}SO₂F)— | −108 |
| —CH₂CF₂—CH₂CF₂—CF₂CF(R_{F})— | −110 |
| —OCF₂CF(CF₃)OCF₂CF₂SO₂F— | −112 |
| —CH₂CF₂—CH₂CF₂—CF₂CH₂— | −113 |
| —CH₂CF₂—CF₂CH₂—CH₂CF₂— | −116 |
| —CH₂CF₂—CF₂CF(C₃H₆CN)—CH₂CF₂— | −119 |
| —CF2CF(OR_{F}—SO₂F)—CF₂CF(C₃H₆CN)—CH₂—CF₂— | −120 |
| —CH₂CF₂—CF₂CF(OR_{F}SO₂F)—CH₂CF₂— | −122 |
| —CH₂CF₂—CF₂CF(OR_{F}SO₂F)—CH₂CF₂— | −125 |
| —CH₂CF₂—CF₂CF(OR_{F}SO₂F)—CF₂CH₂— | −127 |
| —OCF₂CF(CF₃)OC₂F₄SO₂F | −144 |
| —CH₂CF₂—CF₂CF(C₃H₆CN)—CH₂CF₂— | −161 to −165 |
| —CH₂CF₂—CF₂CF(C₃H₆CN)—CF₂— | −178 to −182 |

Crosslinking of Fluorosulphonated Nitrile Elastomers

The elastomers of this invention can be crosslinked by using tetraphenyltin or silver oxide, which by action on the nitrile groups, lead to triazine rings. Such systems are well known, such as those described in the reviews Prog. Polym. Sci. 14 (1989) 251 and 26 (2001) 105, as well as in the chapter “Perfluoroelastomers and their Functionalizaton” in the book “Macromolecular Design of Polymeric Materials” (1997). The vulcanization of these elastomers can also be carried out using ionic methods or by radiation or by electron bombardment as described in the article by Lyons, Chapter 18, pages 335-347 of the book “Modern Fluoropolymers” (1997) (edited by J. Scheirs).

Copolymers with such compositions can be used in the production of O-rings, pump housings, diaphragms having very good resistance to fuels, to gasoline, to t-butyl methylether, to alcohols, to motor oils and to strong acids (e.g. HCl, HNO₃ and H₂SO₄), combined with good elastomeric properties, in particular very good resistance to low temperatures. These copolymers also have the advantage of being crosslinkable in the presence of the agents that are traditionally used.

### EXAMPLES

The following examples, which are given in order to better illustrate the present invention, should in no way be interpreted as constituting any limitation to the scope of said invention.

### Example 1

### Synthesis of 1,2-dichloro-1-iodotrifluoroethane

A Carius tube (interior diameter: 78 mm, thickness: 2.5 mm and length: 310 mm) containing a magnetic bar, 175.5 g (1.08 mols) of iodine monochloride (ICl), 1.1 g (0.006 mol) of benzophenone and 150 g of methyl chloride are cooled in a liquid nitrogen/acetone mixture (−80° C.). After having carried out 3 vacuum/nitrogen cycles, 131 g (1.12 moles) chlorotrifluoroethylene (CTFE) are added. The tube is sealed, then reheated gradually to ambient temperature, then the solution is stirred under ultraviolet light (UV, Philips HPK 125 W mercury vapour lamp) for 6 hours. After processing in the presence of sodium thiosulphate, then drying on magnesium sulphate and evaporation of the methylene chloride, the distillation yields 204.9 g of pink liquid (boiling point: 99-101° C.) with a yield of 68%. The product obtained is a mixture of two isomers: 1-iodo-1,2-dichlorotrifluoroethane (92%) and 1,1-dichloro-2-iodotrifluoroethane (8%).

¹⁹F NMR of the first isomer (CDCl₃) δ: system ABX δ(F₂ₐ)=−62.31; δ(F_{2b})=−65.25; δ(F₁)=−72.87; J(F₂ₐ-F_{2b})=163.9 Hz; J(F₂ₐ-F₁)=14.4 Hz; J(F_{2b}-F₁)=15.6 Hz.

¹⁹F NMR of the second isomer (CDCl₃) δ: system A₂X δ(F₁)=−55.60; δ(F₂)=−67.65; J(F₁-F₂)=14.4 Hz.

### EXAMPLE 2

### Addition of 1,2-dichloro-1-iodotrifluoroethane to allyl cyanide

279.0 g (1.00 mole) of ClCF₂CFClI and 70.5 g (1.05 mole) of allyl cyanide are introduced into a flask with three necks, equipped with a condenser and a nitrogen scavenging system. The reaction mixture is gradually heated to 80° C. and 2.48 g (15 mmole) of AIBN that has been previously recrystallized in methanol is added. The reaction mixture is stirred for 2 hours and a sampling is carried out, followed by another addition of AIBN (2.50 g; 15.2 mmole). The reaction is then maintained at 80° C. and its progress is tracked using gas phase chromatography (CPV). After 6 hours of reaction, the CPV chromatogram of the unpurified reaction mixture shows the almost complete conversion of the 1,2-dichloro-1-iodotrifluoroethane. The global yield is about 90%. After distillation of the excess of allyl cyanide that has not reacted, 310 g of a dark residue is recovered and analyzed using IRTF (IR Nicolet 510 P) and with NMR (Bruker 200 MHz).

IRTF (KBr, cm⁻¹): 2,936.0 (ν_{C-C}); 2,270.8 (ν_{CN}); 1,450 (ν_{CH2}); 1,248-1,293 (ν_{CH2}); 1,079.9 and 1,265 (ν_{C-F}); 705.2 (ν_{C-Cl}); 502.3 (ν_{C-l}).

### NMR Characterization of 3-iodo-5,6-dichloro-5,6,6-trifluoro-hexane nitrile

¹H NMR (CDCl₃) δ: 2.5-3.4 (m, CFClCH₂ and CH₂CN, 4H); 4.45 (m, CHI, ¹H).

¹⁹F NMR (CDCl₃) δ: −68 (system AB, ClCF₂, 2F); −118.5 and −122.5 (part X of a system ABX, the two peaks being attributed to the two diastereoisomers, CFCl—, IF).

### Example 3

### Synthesis of 5,6-dichloro-5,6,6-trifluoro-hexane nitrile

Into a two-necked flask equipped with a cooler previously saturated with argon and containing an agitated solution made up of 108.1 g (0.312 mole) of the fluoro-iodated nitrile described above and 100 g of anhydrous THF, 100.0 g (0.344 mole) tributyltin hydride is added drop by drop in argon atmosphere at 0-5C. After addition, the stirred reaction mixture is gradually heated to ambient temperature (1 hour) then heated to 40° C. over 2 hours. After cooling, the unpurified reaction mixture is distilled. The THF is first eliminated, then a yellow liquid fraction (60.4 g) corresponding to the fluorinated hexane nitrile no longer containing iodine is obtained. The yield is 88%. Boiling point=104-109° C./22 mm Hg.

### Characterization of 5,6-dichloro-5,6,6-trifluoro-hexane nitrile (ClCF₂CFClCH₂CH₂CH₂CN)

IRTF (KBr, cm⁻¹): 2,951.5 (ν_{CC}); 2,271.0 (ν_{CN}); 1,250-1,295 (ν_{CH2}) 1,050-1,215 (ν_{CF}); 703. 5 (ν_{CCl}).

NMR of ¹H (CDCl₃) δ: 2.05 (q, ³J_{HH}=7.0 Hz, CH₂CH₂CN, 2H); 2.2-2.5 (m, CFClCH₂CH₂, 4H).

NMR of ¹⁹F (CDCl₃) δ: −68.5 (system AB, ClCF₂, 2F); −120.5 (m, CFCl, 1F).

### EXAMPLE 4

### Synthesis of 5,6,6-trifluoro-5-hexene nitrile (F—CN) (CF₂═CF—CH₂CH₂CH₂CN)

Into a two-necked flask equipped with a coolerand containing an stirred solution made up of 21.34 g (0.326 mole) of zinc, 6.62 g (0.048 mole) of ZnCl₂ and 130 g DMF, a solution made up of 65.3 g (0.44 mol) of 5,6-dichloro-5,6,6-trifluoro-hexane nitrile in 40 g of DMF is added drop by drop at 40° C. After addition, the stirred unpurified reaction mixture is heated to 90° C. and kept at this temperature for 4 hours. After cooling, the unpurified reaction mixture is treated with an acid solution (HCl 10%) then neutralized with NaHCO₃ and washed with water. The extraction with ClCF₂CFCl₂ (F-113) followed by drying on MgSO₄ yielded, after distillation of the F-113, to 18.6 g of F₂C═CFC₃H₆CN, which corresponds to a yield of 42%. A violet liquid is obtained. Boiling point=76-78° C./21 mm Hg.

IRTF (KBr, cm⁻¹): 2,945.7 (ν_{cc}); 2,270.5 (ν_{CN}); 1,799.81 (ν_{=CF}); 1,448.7 (ν_{CH2}) 1,294-1,246 (ν_{CH2}); 1,028-1,188 (ν_{CF}).

NMR ¹H (CDCl₃) δ: 2.45 (t, ³JHH=6.5 Hz, CH₂CN, 2H); 2.35 (dddt, ³JHFC=22.5 Hz, ⁴J_{HFa}=2.4 Hz, ⁴J_{HFb}=4.0 Hz, ³J_{HH}=6.8 Hz, CH₂CF═, 2H); 1.85 (q, ³J_{HH}=6.9 Hz CH₂CH₂CN).

NMR ¹⁹F (CDCl₃) δ: −103.5 (dd, ²J_{FaFb}=82.8 Hz, ³J_{FaFc}=33.3 Hz; Fa); −124.0 (ddq, ²J_{FbFa}=82.8 Hz, ³J_{FbFc}=114.3 Hz, ⁴J_{FbH}=3.7 Hz; F_{b}); −175.5 (ddt, 3J_{FcFb}=114.2 Hz, ³J_{FcFa}=33.1 Hz, ³J_{FcH}=21.0 Hz; F_{c});

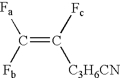

### EXAMPLE 5

### Synthesis of fluorosulphonated nitrile elastomers by radical copolymerization VDF/F₂C═CFC₃H₆CN/CF₂═CFOCF₂CF(CF₃)OC₂F₄SO₂F

In the case of example 5, (see Table 1), we used a 160 ml reactor made of Hastelloy, equipped with two valves, with a rupture disc and a gauge, into which was introduced 4.6 g (0.030 mole) of CF₂═CFC₃H₆CN, 28.4 g (0.062 mole) of PFSO₂F, i.e. CF₂═CFOCF₂CF(CF₃)OC₂F₄SO₂F, 0.22 g (1.5×10⁻³ mole) of tertiobutyl peroxide and 30.0 g of acetonitrile. The reactor is closed, placed in a vacuum and cooled in a liquid acetone/nitrogen mixture. Once the temperature reaches −80° C., 14.0 g (0.218 mole) of vinylidene fluoride is introduced. The reactor is allowed to come back to ambient temperature, then it is heated to 135° C. over 15 hours. After cooling in ice, the reactor is degassed and 2.8 g of VDF that had not reacted was salted out (the mass yield of VDF is 80%). The characterization by ¹⁹F NMR of the unpurified reaction mixture shows that 80% of the sulphonated monomer has reacted (the presence of the characteristic signal centered at −138.5 ppm gives evidence of the presence of the monomer that has not completely reacted). The acetonitrile is partially evaporated, then the copolymer is precipitated by addition, drop by drop, into 200 ml of vigorously stirred cold pentane. The copolymer sticks to the walls of the Erlenmeyer flask and after decanting, separation and drying in a vacuum at 80° C. to a constant weight, 38 g of very viscous brown-amber product is obtained. The mass yield is 75%. The ¹⁹F NMR spectrum makes it possible to know without ambiguity the molar percentages of the three comonomers using the characteristic signals of the different fluorinated groups contained in the units making up the VDF (72 mol-%) of PFSO₂F (25 mol-%) and nitrile monomer F—CN (3.0 mol-% (see Table 2). The chemical shifts in ¹⁹F NMR of the fluorinated groups of the copolymers (see Table 2) have been determined without ambiguity from all of the polymers obtained, for which the experimental details and the results are given in Table 1. Differential calorimetric analysis (DSC), using a Perkin Elmer Pyris 1 instrument calibrated to indium and to octadecane, was carried out using a sample of around 15 mg with three heating cycles from −100° C. to +165° C. (at 40, then 20° C./min⁻¹)/cooling from +165° C. to −100° C. (at 320° C. min⁻¹). The results on the copolymers have led to evidence of a single glass transition temperature (T_{g}) corresponding to the inflection point of the enthalpy jump. The second and third cycles yielded reproducible values of T_{g}. Thus the DSC analysis has shown the absence of a peak attributed to fusion, but the presence of an enthalpy jump attributed to a single glass transition temperature. The T_{g} is −31° C.

Thermogravimetric analyses (TGA) carried out using a TGA 51-133 device, Texas Instruments, in air, with a heating rate of 10° C. min⁻¹, have shown that the copolymer lost around 5% of its mass at 285° C. The ¹⁹F NMR analysis characterizing the different chemical shifts of the various fluorinated groups are given in Table 2.

IRTF (KBr, cm⁻¹): 2,948.7 (ν_{CC}); 2,266.8 (ν_{CN}); 1,464.6 (ν_{SO2F}); 1,445.3 (ν_{CH2}); 1,113-1,210 (ν_{CF}).

### Example 6

### Synthesis of fluorosulphonated nitrile elastomers using radical copolymerization VDF/F₂C═CFC₃H₆CN/CF₂═CFOCF₂CF(CF₃)OC₂F₄SO₂F

The synthesis is carried out according to the experimental process described in a detailed manner in example 5 above, with the exception of the masses of monomers VDF, PFSO₂F and F—CN, which are more important and with the exception of the initiator, which is of a different nature (as well as the usage temperature of the said initiator) as is indicated in column C₀ of comparative Table 1. The infrared spectrum is analogous to that in example 5.

### Example 7

### Synthesis of fluorosulphonated nitrile elastomers using radical copolymerization

VDF/F₃C═CFC₃H₆CN/CF₂═CFOCF₂CF(CF₃)OC₂F₄SO₂F

The synthesis is carried out according to the experimental process described in a detailed manner in example 5 above, with the exception of the masses of monomers VDF, PFSO₂F and F—CN, which are more important such as it is indicated in comparative Table 1. In addition, the masses of monomers PFSO₂F and F—CN are more important than those used in example 6.

The infrared spectrum is analogous to that in example 5.

### EXAMPLE 8

### Crosslinking of the fluorosulphonated nitrile copolymers

10.05 g of the copolymer described in example 5 is dissolved in 30.5 g of acetone. 3.2 g of carbon black and 0.53 g (1.3 mmole) of tetraphenyltin (Aldrich) are added to it. Once the solution is homogeneous, the acetone is evaporated, then the viscous residue is spread out in a mould, located between two sheets of PTFE, pressed (pressure 20 bars) at 175° C. for 2 hours then at 200° C. for 24 hours, and finally at 220° C. for 12 hours. The film obtained is very thin (15 to 20 μm), homogeneous and insoluble in all organic solvents and hydrocarbons, as well as in concentrated HCl and H₂SO₄.

IRTF (KBr, cm⁻¹): 2,962.8 (VCH); 1,580 and 1,502 (ν_{C=N}, triazine); 1,464.2 (ν_{SO2F}); 1,110-1,207 (ν_{CF}).

1. A compound of formula I:
Z₂C═CWX(CY₂)ₙCN (I)
wherein
X is an atom of oxygen or no atom;
Y is an atom of hydrogen or fluorine;
Z is an atom of hydrogen or fluorine;
W is an atom of hydrogen or fluorine or a CF₃ group; and
n is a natural integer between 0 and 10 inclusively,
provided that at least one of the following is true:
Y is a hydrogen atom,
W is a CF₃ group;
and excluding a compound of formula F₂C—CF(CH₂)₃CN.

2. The compound of claim 1, of formula II:
F₂C═CF(CH₂)ₙCN (II)
wherein n is a natural integer between 0 and 10 inclusively.

3. A process of preparation of a fluorinated copolymer using radical copolymerization, comprising:
reacting a compound of claim 1 with
a compound of formula III₁
F₂C═CFOR_{F1} (III₁)
wherein R_{F1} is a linear or branched group of the formula CₙF₂ₙ₊₁, wherein n is a natural integer varying from 1 to 10;
or with
a compound of formula III₂
F₂C═CFOR_{F2}-G (III₂)
wherein R_{F2} is a linear or branched group of the formula (CF₂CFX′)_{y}[O(CF₂)ₗ]ₘ, wherein X′ is a fluorine atom or a CF₃ group; and
wherein y, l, and m are natural integers between 1 and 5 inclusively, 1 and 4 inclusively, and 0 and 6 inclusively, respectively; and
wherein G is a functional group SO₂F, CO₂H, CO₂R, wherein R is the group CₚH₂ₚ₊₁ wherein p is a natural integer varying from 0 to 5; or a functional group P(O)(OR′), wherein R′ is a hydrogen atom or a C₁-C₅ alkyl group.

4. The copolymerization process of claim 3, comprising:
reacting a compound of formula II′:
F₂C═CF(CH₂)₃CN (II′)
with a compound of formula III₁ or a compound of formula III₂, in such a way as to obtain a random copolymer of formula IV: 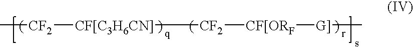
wherein R_{F} is the group R_{F1} or R_{F2}, wherein group G is absent when R_{F} is R_{F1}, wherein q, r, and s are, independently, natural integers such that the ratio q/r various from 1 to 30 and such that s varies from 20 to 300.

5. A copolymerization process, comprising:
reacting a compound of formula II′
F₂C═CF(CH₂)₃CN (II′)
with
a compound of formula III₁
F₂C═CFOR_{F1} (III₁)
wherein R_{F1} is a linear or branched group of the formula CₙF₂ₙ₊₁,
wherein n is a natural integer varying from 1 to 10;
or with
a compound of formula III₂
F₂C CFOR_{F2}-G (III₂)
wherein R_{F2} is a linear or branched group of the formula (CF₂CFX′)_{y}[O(CF₂)ₗ]ₘ, wherein X′ is a fluorine atom or a CF₃ group;
and wherein y, l, and m are natural integers between 1 and 5 inclusively, 1 and 4 inclusively, and 0 and 6 inclusively, respectively;
and wherein G is a functional group SO₂F, CO₂H, CO₂R, wherein R is the group CₚH₂ₚ₊₁ and wherein p is a natural integer varying from 0 to 5;
or a functional group P(O)(OR′), wherein R′ is a hydrogen atom or a C₁-C₅ alkyl group,
and with
a compound of formula V
FCX═CYZ (V)
wherein X, Y, and Z are, independently, atoms of hydrogen, fluorine, chlorine, or a group of formula CₙF₂ₙ₊₁, wherein n is 1, 2, or 3, with the proviso that X, Y, and Z are not all fluorine atoms,
in such a way as to obtain a random copolymer of formula VI: 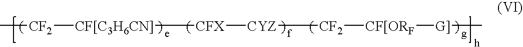
wherein R_{F} is R_{F1} or R_{F2}, group G being absent when R_{F} is R_{F1}, and wherein e, f, g, and h are, independently, natural integers such that the ratio f/e varies from 5 to 50, such that the ratio f/g varies from 1 to 20 and such that h varies from 10 to 250, wherein R_{F1} is a linear or branched group of the formula CₙF₂ₙ₊₁, wherein n is a natural integer varying from 1 to 10,
wherein R_{F2} is a linear or branched group of the formula (CF₂CFX′)_{y}[O(CF₂)ₗ]ₘ, wherein X′ is a fluorine atom or a CF₃ group; and wherein y, l, and m are natural integers between 1 and 5 inclusively, 1 and 4 inclusively, and 0 and 6 inclusively, respectively.

6. The copolymerization process of claim 4, wherein the reaction is carried out in batch.

7. The copolymerization process of claim 4, wherein the reaction is carried out in emulsion, in microemulsion, in suspension, or in solution.

8. The copolymerization process of claim 4, wherein the reaction is initiated in the presence of at least one organic radical initiator.

9. The copolymerization process of claim 4, wherein the reaction is carried out in the presence of at least one organic radical initiator selected from the group consisting of alkyl peroxides, peresters, percarbonates, alkyl peroxypivalates, and diazo compounds.

10. The copolymerization process of claim 4, wherein the reaction is carried out in the presence of at least one of the following:
a) at least one peroxide selected from the group consisting of t-butyl peroxide, t-butyl hydroperoxide, t-butyl peroxypivalate, and t-amyl peroxypivalate; and/or
b) at least one benzoyl peroxide; and
c) at least one t-butyl cyclohexyl peroxydicarbonate.

11. The copolymerization process of claim 9, wherein the concentration of at least one of peroxide, perester and percarbonate in the reaction medium is such that the initial molar ratio between initiator and the monomers ([initiator]₀/[monomers]₀) is between 0.1 and 2%, wherein the initator is t-BuO-OtBu or tBuO-OC(O)tBu, and wherein the monomers are compounds selected from the group consisting of formulas I, II, III₁, III₂, II′, and V; wherein the expression [initiator]₀ expressed the initial molar concentration of initiator and the expression [monomers]₀ expresses the total initial concentration of monomers.

12. The copolymerization process of claim 4, wherein the reaction is carried out:
in the presence of t-butyl peroxypivalate and at a reaction temperature between 70 and 80° C.; or
in the presence of t-butyl peroxide and at a reaction temperature between 135 and 145° C.

13. The copolymerization process of claim 4, wherein the reaction is carried out in solution in the presence of at least one organic solvent.

14. The copolymerization process of claim 13, wherein the organic solvent is perfluoro-n-hexane, acetonitrile, or a mixture thereof.

15. The copolymerization process of claim 13, wherein the solvent content of the reaction mixture is such that the initial mass ratio between the solvent and the monomers is between 0.5 and 1.5.

16. The copolymerization process of claim 4, wherein the compound of formula III₂ is perfluoro(4-methyl-3,6-dioxaoct-7-ene)sulphonyl fluoride and wherein the compound of formula V is vinylidene fluoride.

17. A fluorinated polymer obtained by the process of claim 3.

18. A fluorosulphonated nitrile copolymer obtained by the process of claim 3.

19. The fluorosulphonated nitrile copolymer of claim 18, comprising:
from 1 to 20 mol % of 5,6,6-trifluoro-5-hexene nitrile (F—CN);
from 20 to 30 mol % of perfluoro(4-methyl-3,6-dioxaoct-7-ene)sulphonyl fluoride (PFSO₂F); and
from 65 to 79 mol % vinylidene fluoride (VDF).

20. The fluorosulphonated nitrile copolymer of claim 19, comprising:
from 2 to 14 mol % of 5,6,6-trifluoro-5-hexene nitrile (F—CN);
from 20 to 30 mol % of perfluoro(4-methyl-3,6-dioxaoct-7-ene)sulphonyl fluoride (PFSO₂F); and
from 66 to 78 mol % vinylidene fluoride (VDF).

21. The fluorosulphonated nitrile copolymer of claim 21, comprising the following groups:
—SO₂F;
—OCF₂CF(CF₃)OCF₂CF₂SO₂F;
tBuO-CF₂CH₂—;
—CH₂CF₂—CH₂CF₂—CH₂CF₂—;
—CF₂CF(RF)—CH₂CF₂—CH₂CF₂—;
—CF₂CF(RF)—CH₂CF₂—C H₂CF₂—CF₂CF(RF)—;
—CH₂CF₂—CH₂CF₂—CF₂CH₂—;
—CF₂CF(ORFSO₂F)—CH₂CF₂—CF₂CF(ORFSO₂F)—;
—CH₂CF₂—CH₂CF₂—CF₂CF(RF)—;
—OCF₂CF(CF₃)OCF₂CF₂SO₂F—;
—CH₂CF₂—CH₂CF₂—CF₂CH₂—;
—CH₂CF₂—CF₂CH₂—CH₂CF₂—;
—CH₂CF₂—CF₂CF(C₃H₆CN)—CH₂CF₂—; —CF₂CF(ORF—SO₂F)—CF₂CF(C₃H₆CN)—CH₂—CF₂—;
—CH₂CF₂—CF₂CF(ORFSO₂F)—CH₂CF₂—;
—CH₂CF₂—CF₂CF(ORFSO₂F)—CH₂CF₂—;
—CH₂CF₂—CF₂CF(ORFSO₂F)—CF₂CH₂—;
—OCF₂CF(CF₃)OC₂F₄SO₂F;
—CH₂CF₂—CF₂CF(C₃H₆CN)—CH₂CF₂—; and
—CH₂CF₂—CF₂CF(C₃H₆CN)—CF₂—;
associated, respectively, with the following chemical shifts, expressed in ppm, in ¹⁹F NMR:
+45;
−77 to −80;
−83;
−91;
−92;
−93;
−95;
−108;
−110;
−112;
−113;
−116;
−119;
−120;
−122;
−125;
−127;
−144;
−161 to −165; and
−178 to −182.

22. A process of preparation of a fluorosulphonated nitrile elastomer, wherein the copolymer obtained in claim 3 is subjected to a crosslinking step.

23. The process of claim 22, wherein the crosslinking step is carried out in the presence of tetraphenyltin or silver oxide in proportions varying from 0.1 to 10 parts by weight for 100 parts by weight of fluorosulphonated nitrile copolymer, and wherein said crosslinking step is carried out at a pressure of 20 bars at 175° C. for two hours, at 200° C. for twenty-four hours, and at 220° C. for 12 hours.

24. A fluorosulphonated nitrile elastomer obtained by the process of claim 22.

25. The fluorosulphonated nitrile elastomer of claim 24, wherein said elastomer has a very low glass transition temperature (T_{g}), wherein said glass transition temperature is measured according to the standard ASTM E-1356-98, and wherein said glass transition temperature is between −43° and −22° C.

26. The fluorosulphonated nitrile elastomer of claim 24, wherein said elastomer has an inherent viscosity, measured according to the method ASTM D-2857-95, between 0.9 and 2.0 mL/g.

27. The fluorosulphonated nitrile elastomer according to claim 25, wherein said elastomer has a thermostability measured by thermogravimetric analysis of up to 297° C. in air at 10° C./min, wherein a loss of mass of 5% is measured.

28. A cross-linking process for cross-linking sulfonyl groups of a sulphonated polymer of claim 24 comprising:
bringing the polymer into contact with a crosslinking agent to form crosslinking bonds between two sulphonyl groups from adjacent polymer chains, wherein at least a fraction of the bonds formed during crosslinking have an ionic charge.

29. A method of manufacturing membranes, polymer electrolytes, ionomers, or fuel cell components comprising using one or more fluorosulphonated cross-linkable nitrile elastomers of claim 18 to manufacture membranes, polymer electrolytes, ionomers, or fuel cell components.

30. A method for obtaining gaskets, O-rings, radiator hoses, tubes, pump bodies, diaphragms, or piston heads comprising using one or more fluorosulphonated cross-linkable nitrile elastomers of claim 18 to manufacture gaskets, O-rings, radiator hoses, tubes, pump bodies, diaphragms, or piston heads.

31. A method for manufacturing plastics comprising using one or more fluorosulphonated cross-linkable nitrile elastomers of claim 18 to manufacture plastics.
